(19) United States
(12) Patent Application Publication
(10) Pub. No.: US 2004/0102764 A1
(43) Pub. Date: May 27, 2004

(54) Laser ablation

(76) Inventor: Peter Balling, Arhus (DK)

Correspondence Address: NIXON PEABODY, LLP, 401 9TH STREET, NW, SUITE 900, WASINGTON, DC, 20004-2128, US

(21) Appl. No.: 10/416,587

(22) PCT Filed: Nov. 6, 2001
(86) PCT No.: PCT/DK01/00730

(30) Foreign Application Priority Data
Nov. 13, 2000 (DK) PA 2000 0160
Jun. 8, 2001 (DK) PA 2001 00890

(51) Int. Cl. A61B 18/20; B23K 26/02

(52) U.S. Cl. 606/5; 219/121.83; 606/10

(57) ABSTRACT

The invention provides a method for measuring in situ the amount of material removed by laser ablation with ultrashort laser pulses. The method relies on the geometrical information provided by the backscattered light from the ablating laser. The temporal structure of the backscattered laser light is used to provide an accurate measure for the depth of the ablated area, since the round-trip time for the short laser pulses uniquely determines the distance to the object under illumination. For femtosecond laser pulses a depth resolution of a few micrometers can be achieved. According to the invention, imaging of the backscattered light from a single ablating pulse provides all the information necessary to derive a cross-sectional profile across the ablated region.

### FIELD OF THE INVENTION

This invention relates to laser ablation with ultrashort laser pulses. It is applicable both within laser ablation of small structures (micromachining) in metals, insulators, and semiconductors and in biological tissue (laser therapy).

### BACKGROUND OF THE INVENTION

Material removal by lasers has become important in various applications within machining and medical treatments. As discussed in international patent application WO99/67048, ultrashort laser pulses, which are focused onto a surface, have the ability to remove material at very low energies and with very small thermal effects on the region surrounding the ablated area. This has important implications with respect to the applications of the ablation. For example, this allows ablation of very small structures, micromachining, and the machining of very thin objects.

Laser ablation has a strongly non-linear dependence on the intensity of the used laser light. In U.S. Pat. No. 5,656,186, this feature has been used to devise a method for the generation of structures that are smaller than the laser spot size. From this patent, it is known to collect emission from the plasma target and relating the intensity of this emission to the amount of material ablated. However, no information is obtained by this method about the depth of the ablated region.

In German patent publication DE19736110, it is emphasised that the unwanted effects of having a laser focus in front of the sample can be eliminated by using diffractive optics for the imaging onto the sample.

In international patent application WO9955487, the importance of the direction of the laser polarisation relative to the cutting direction is pointed out. Similarly, it is discussed in German patent publication DE19744368 that rotation of the laser polarisation or the application of circularly polarised light can eliminate the unwanted geometrical effects caused by laser ablation with a linearly polarised light field.

Another potentially important area of applications for ultrashort-pulse laser ablation is within laser therapy, where the reduced heat deposition minimises unwanted biological effects, this is described in U.S. Pat. No. 5,720,894.

The high lateral (transverse) resolution in the machining with ultrashort laser pulses is a consequence of the reduced heat deposition, which minimises melting of the surrounding regions. In addition, the vertical (depth) resolution can also be high, since all the light is absorbed in a thin surface layer (the skin depth) with practically no heat diffusion during the laser pulse. This high accuracy has been demonstrated in a variety of scientific publications, and the manufacturing of three-dimensional structures is feasible.

The previously mentioned studies of laser-machined structures have applied high-resolution microscopy from, e.g., a scanning electron microscope, to image the produced structures. While this is an extremely valuable diagnostics for characterising the structures, it is not a method suited for on-the-fly (i.e. during machining) characterisation. However, the possibility to retrieve geometrical information during the ablation process is highly desirable as this would allow a variety of very detailed control procedures based on, e.g., a feed-back loop.

In U.S. Pat. 5,744,780, long laser pulses reflected from a laser machined sample are used to examine the progress of material removal on the workpiece.

Laser ranging by ultrashort laser pulses is an established technique, which is described in text books on ultrashort laser pulses, see, e.g., Diels and Rudolph Ultrashort laser pulse phenomena (Academic Press 1996). The necessary time-resolution is obtained by various optical gating techniques. The optical Kerr effect can be used to select a specific flight time and thus a given distance. Distance determinations based on second-harmonic generation have been described in U.S. Pat. Nos. 5,585,913 and 5,489,984. By using an appropriate optical arrangement, it is possible to obtain a two-dimensional image at a specific distance, see, e.g., Yan et al., Applied Optics 31, 6869 (1992). In U.S. Pat. No. 5,710,429 and references herein, this technique is investigated as a means for performing imaging through highly-scattering media.

The imaging techniques described above fall in two main categories. Either the distance is sampled in a single spot, which then for some applications can be scanned across a sample. Alternatively a full two-dimensional image at a given distance is acquired in a single shot. In both cases, the distance-co-ordinate (depth) must be scanned to obtain three-dimensional information.

According to the above description of prior art, it is known to use laser ablation in various circumstances, and it is also known to measure depth profiles on a sample with laser radiation. However, a combination of laser ablation with depth profile measurements is not known.

It would be desirable to know a method where depth information can be obtained during laser ablation. Also, it would be desirable if the depth of the structure and spatial information could be obtained with the same light pulse during laser ablation, especially if this information is provided with the same laser pulse that causes the ablation, because then, obtaining the depth and/or spatial information would not consume any time and hence would not affect the ablation rate. To provide such a method and system is the purpose of the invention.

### SUMMARY OF THE INVENTION

This object is achieved with a method according to claim 1.

The invention uses the temporal and preferably also spatial properties of the backscattered light from the laser ablation process to provide information about the resulting geometrical structure.

The depth information is obtained by performing a high-resolution determination of the flight time for the ultrashort laser pulses impinging on the sample. The backscattered light can be of duration much longer than the incoming laser pulse but a certain fraction of the light will take the shortest possible trajectory to the sample and back. By gating the detection with a time resolution similar to the pulse duration, it is possible to select this (ballistic) part of the light and thus to extract the exact distance to the sample in the current geometry. The resolution in the distance measurement is determined by the laser pulse duration, and for an ultrafast laser pulse a depth resolution of a few micrometers can be obtained.

Ablating material from a surface with a laser pulse and at the same time with the same pulse obtaining geometrical information is a great advantage, because only one laser and the same optical set-up is used. Furthermore, no additional time is consumed by obtaining the needed geometrical information.

Metallic samples have a high reflectivity, and also semiconductors tend to show very high transient reflectivity when subjected to an intense laser pulse. For other media (insulators or biological tissue) scattering or reflection from the sample is possible only, if a plasma is generated on the surface of the sample. Especially for these media, it is a great advantage to obtain geometrical information during the ablation. The geometry of the original surface is obtained with high accuracy, because the radiation reflecting plasma will not expand significantly during the laser pulse.

In a practical implementation of the invention, the laser light is split in two parts, where one part is directed onto the sample to perform the ablation, while the other part is sent through a variable distance, a so-called delay line, and is used for timing a so-called optical gate. An optical gate is a device which works much like a mechanical shutter, but with an ultra-short opening time that can be as short as the duration of the timing pulse.

Preferably, the backscattered light is collected by the same optics as is used to focus or image the laser light onto the sample. This allows for a simple design while permitting a large numerical aperture of the optics used for collecting the light. The latter has two advantages. First, it ensures a high collection efficiency and hence maximum sensitivity in the distance measurement. Second, a high numerical aperture is needed to provide a high lateral resolution in an imaging geometry, as will be explained further below.

The optical gate may be based on non-linear frequency mixing. A time-correlated laser pulse, the timing pulse, and the backscattered light are directed onto a non-linear medium (e.g. a non-linear crystal) and the delays are adjusted so that the laser pulse impinges on the medium together with the fastest (ballistic) part of the backscattered radiation. When both light fields are present, the non-linear mixing produces a new light field at, e.g., twice the frequency. Since both incoming light fields need to be present to generate the new light field and the timing pulse is very short, the generated field reflects the backscattered intensity at a very well-defined flight time. This is directly related to a well-defined depth and thus ensures a high resolution.

In a specific embodiment of the invention using an optical gate based on non-linear mixing, the mixing is performed in a non-collinear geometry. This reduces the background and thus increases sensitivity. In addition, by selecting an appropriate geometry, the spatial distribution of the generated light field reflects the temporal distribution of the backscattered light. This technique is similar to that applied in single-shot auto-correlators.

In another embodiment of the invention, the backscattered light is sent through an optical transmission line, which images (preferably magnifies) the interaction region on the sample onto or through the optical gate. If the light transmitted through the gate (e.g. the field of twice the frequency in a specific embodiment) is further imaged onto a detector, the light carries information about the two dimensional cross-sectional geometry of the ablated area in addition to the obtained depth information. Thus, by scanning the gating time of the optical gate, it is possible to obtain a three dimensional image of the ablation region.

In a further embodiment of the invention, non-linear frequency mixing in a non-collinear geometry is used in combination with imaging of the ablation region onto the non-linear crystal. Thereby, a pattern is produced in the crystal, where the image of the pattern in one direction provides temporal information about the backscattered light, which is related to the surface height of the sample, and the perpendicular direction provides information about the cross-sectional geometry along a specific axis on the sample. As will become clear from the various examples of use listed in the detailed description, this amount of geometrical information is exactly what is needed for controlling the ablation process in most cases.

### BRIEF DESCRIPTION OF THE DRAWINGS

FIG. 1 illustrates the backscattering of the laser light from the sample,

FIG. 2 shows a schematic drawing of an optical set-up according to the invention,

FIG. 3 gives a detailed view of the optical gate and imaging system in the specific embodiment, where non-linear mixing is applied,

FIG. 4 shows images of the light transmitted through the optical gate according to FIG. 3 and associated cross-sectional profiles,

FIG. 5 shows the ablation depth versus ablation time and the scatter of the measurements,

FIG. 6 shows two images and associated cross-sectional profiles obtained under conditions of translation of the sample during machining (laser milling).

### DETAILED DESCRIPTION OF THE INVENTION

The invention employs time-gated measurement of the backscattered light from laser ablation to produce on-the-fly imaging of the object subjected to ablation.

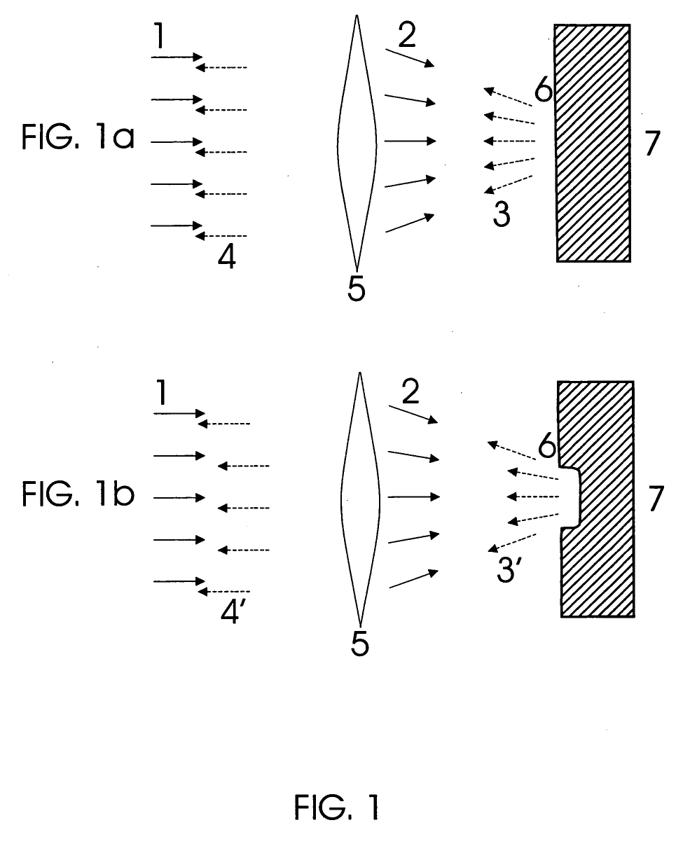

The basic principle of the invention is shown in FIG. 1. Ultrashort light-pulses, indicated by first arrows 1, are focused by lens 5, as indicated by second arrows 2, onto the surface 6 of a sample 7 in order to cause ablation. A part of the incident light is scattered back, as is indicated by third arrows 3, and propagates through the lens 5, as indicated by fourth arrows 4.

The depth information is obtained by performing a high-resolution determination of the flight time for the ultrashort laser pulses impinging on the sample 7. Since the invention is envisioned to be used in the ablating regime, the laser will create a plasma on the surface 6 of the sample 7, and the backscattered light will in general be of a duration much longer than the incoming laser pulse, since the decay of the light is determined by plasma evolution. However, for the purpose of this invention it suffices to note that a certain fraction of the light will take the shortest possible trajectory to the sample 7 and back. By gating the detection with a time resolution similar to the pulse duration, it is possible to select this (ballistic) part of the light and thus to extract the exact distance to the sample 7 in the current geometry. The resolution in the distance measurement is determined by the laser pulse duration, T: If x denotes the distance to the sample and c the velocity of light, the flight time to the sample and back is 2x/c. Thus a temporal resolution of T gives a spatial resolution of cT/2. For an ultrafast laser pulse, T˜10⁻¹⁴s and a depth resolution of a few micrometers is obtained. The principle is similar to that of a radar, which operates with radio waves and uses much longer pulses.

Initially, while the surface is flat as indicated in FIG. 1a, all of the backscattered light traverses the same distance and thus crosses at the same time any plane after the lens 5 (arrows 4 are aligned). In FIG. 1b, the light has ablated material from the surface. Since the radiation propagating to the bottom of the structure formed by ablation travels a longer distance, this part of the backscattered light is now delayed relative to the situation in FIG. 1a, as indicated by arrows 3′.

In fact, an accurate measure of the increase of the flight time of the backscattered light during ablation provides an absolute determination of the ablation depth. In the situation shown on FIG. 1, the outer part of the laser beam is apparently not intense enough to cause ablation. Consequently, the light backscattered from the edges on FIG. 1b still traverses the same distance as in FIG. 1a, as shown by the arrows 4′. An accurate measurement of the relative delay between the central and outer parts of the beam provides the depth of the hole relative to the surface.

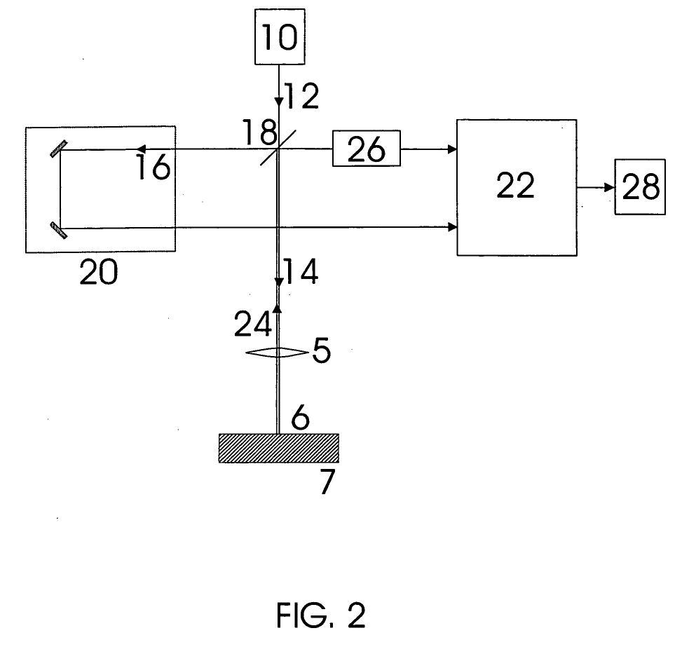

FIG. 2 shows a practical implementation of the invention: The output beam 12 from an ultrashort-pulse laser 10 is split in two parts 14, 16, by a partially reflecting mirror or beam splitter 18. One part 14, the ablating beam, propagates to the sample for performing laser ablation, while the other part 16, the timing beam, is sent through a variable distance delay line 20 to provide the light for the optical gating. The optical set-up must be arranged so that the timing pulse opens the optical gate 22 at the exact time of arrival of the ballistic part of the backscattered light 24. If the response time of the gate 22 is negligible, this implies that the optical path lengths for the ablating beam 14 and the timing beam 16 are exactly equal. In detail, the optical path length from the beam splitter 18, through the focusing lens 5 to the surface 6 of the sample 7, back through the lens 5, reflected from the beam splitter 18, transmitted through imaging lens-system 26 and into the optical gate 22, is exactly the same as the optical path length from the beam splitter 18 through the delay line 20 and into the optical gate 22. The light transmitted through the optical gate 22 is monitored with a detector 28.

In a slightly modified embodiment of the above, the beam splitter 18 is replaced by a so-called polarising beam splitter, which works as a high-reflective mirror for light of one polarisation while it transmits the perpendicular polarisation. By employing a quarter-wave plate on the light path to the sample (e.g. between the polariser 18 and the lens 5, the backscattered light will be linearly polarised at the polarising beam splitter 18 and all the light will be directed towards the optical gate 22. This enhances the sensitivity of the method. In addition, in this geometry, the relative intensity between the ablating beam 14 and the timing beam 16 can be adjusted continuously by rotating the polarisation of the incoming laser beam 12, for example by a half-wave plate.

In a specific embodiment, the optical gate 22 is comprised in a non-linear frequency-mixing scheme: The backscattered 24 and timing 16 light pulses are combined in a non-linear medium, for example a non-linear fluid or a crystal, as a BBO, barium borate, crystal. When both pulses are present in the medium (i.e. at the appropriate delay of the timing pulse) the two light fields mix to produce a new light field at a different frequency, e.g., twice the frequency corresponding to the second-harmonic field. Such an optical gate 22 has negligible response time and thus only opens for a time duration similar to that of the timing light pulse. This time duration is what determines the depth resolution, as mentioned above.

In the preferred embodiment, the two beams are focused non-collinearly onto the same spot on a non-linear crystal, a technique well known in so-called background-free autocorrelation. This separates the background at the second-harmonic beam originating from each of the two light beams independently and leads to a substantially improved sensitivity.

Depending on the specific embodiment of the optical gate 22, it is possible to perform time-resolved imaging of the laser-ablated region. This comprises to insertion of an appropriate imaging lens system 26 in the path of the back-scattered radiation 24 to image the interaction region onto, or through, the optical gate. If the light transmitted through the gate 22 (e.g. the field of twice the frequency in a specific embodiment) is further imaged onto the detector 28, the light carries information about the two-dimensional cross-sectional geometry of the ablated area in addition to the obtained depth information.

Since the light transmitted through the optical gate 22 for fixed gate delay corresponds to a specific flight time, it provides an image associated with a certain height over or depth into the surface. In order to obtain a true three-dimensional image, it is necessary to sample different time delays, which can be achieved by varying the optical path length of the timing beam 16 or the ablation beam 24, for example by scanning the optical delay of the delay line 20.

This, however, may in some circumstances be inconvenient, as the geometrical information needed for a feed-back system requires a scan which takes several laser shots, and during these shots the geometry inevitably changes, which introduces uncertainties of the depth measurement and complicates control of the ablation process. Since laser machining with ultrashort laser pulses requires an amplified laser, the pulse energy available from the laser system, typically between 1 microjoule and 1 millijoule per pulse at a rate of 10 Hertz to a few tens of kilohertz, is normally so high that another approach is foreseen in the invention, which will be explained in the following.

The backscattered 24 and timing 16 light beams are combined as collimated beams with a spot size of several millimetres on the non-linear crystal. A second-harmonic signal only arises from those parts of the crystal where the two beams cross during the (short) pulse duration of the timing pulse. In this way, the temporal information is converted to a spatial pattern and the system can provide information about the signal at a range of time-delays for a single measurement. The technique is known from single-shot autocorrelation methods, where it is applied to measure the pulse-duration of an ultrashort laser pulse in a single shot, as was originally suggested by Jansky et al., Optics Communication 23, 293 (1977), see, e.g., Diels and Rudolph Ultrashort laser pulse phenomena (Academic Press 1996) for a description.

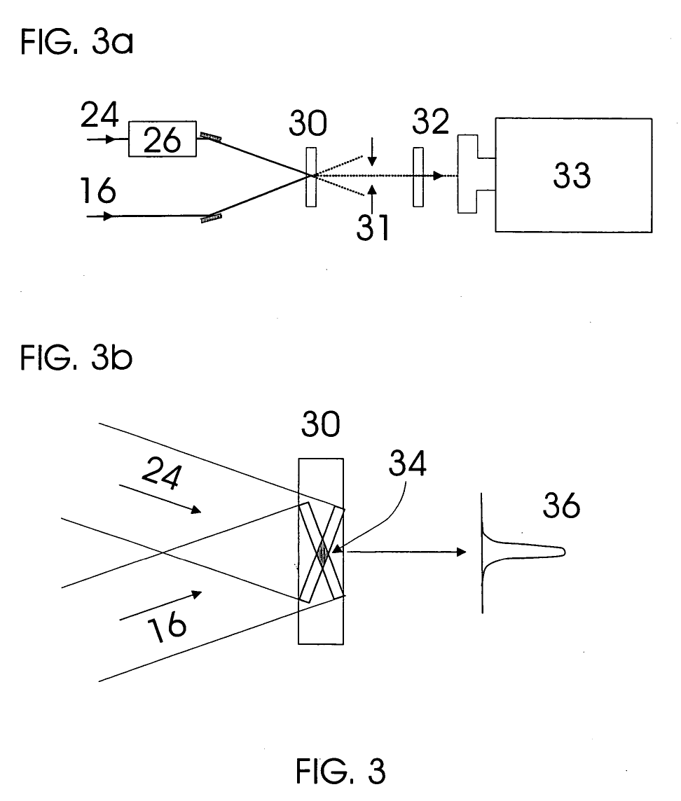

Since one of the spatial co-ordinates reflects temporal information, imaging in this embodiment is limited to only one transverse direction. The embodiment is shown in greater detail in FIG. 3. FIG. 3a shows that the imaging lens system 26 projects the backscattered light 24 to form a two-dimensional image of the interaction region on the non-linear crystal 30, as also illustrated in FIG. 3b. This image is crossed with the timing beam 16 inside the crystal 30. A second-harmonic signal arises from the combined effect of the two beams 16, 24 in those regions 34 of the crystal 30 where the beams 16, 24 overlap. A camera 33, e.g. a charge-coupled device (CCD) camera, collects this light pattern 36. An aperture 31 in front of the camera 33 is used to block the second-harmonic light from the two individual beams 16, 24 (in fact mostly from the intense timing beam 16).

In addition, a combination of filters (32) may be needed, for the following reasons. First, in order to eliminate scattered light from the relatively intense incoming timing beam 16, a filter that blocks the fundamental frequency from the laser is typically applied. Second, depending on the sample 7 under ablation, it may be necessary to attenuate the second-harmonic light pattern 36 to avoid saturation of the camera 33.

As mentioned above, the second-harmonic light generated by the system shown in FIG. 3b, produces a pattern 36, which is formed by the spatial/temporal overlap of the two beams 16, 24 inside the crystal 30. In fact, for typical crossing angles between the two beams 16, 24 (i.e. a few degrees), the timing beam 16 selects only a narrow slice of the image formed from the backscattered light 24, corresponding to the overlap region 34. The width of this slice (or virtual slit) is determined by two contributions. The first contribution is from the transverse distance that the overlap region 34 moves across the image during propagation through the crystal. If the two beams 16, 24 cross at an angle of θ (as measured inside the crystal) and the crystal has a thickness of d, this “walkover” is given by d·sin(θ/2). The second contribution is from the finite laser-pulse duration, T, which gives rise to an effective transverse slit width of cT/sin(θ). For typical geometries and pulses with a time length of 100 femtoseconds, this last term dominates and results in an effective width of the slice (or slit) around a few hundreds of micrometers. Since the imaging system 26 is typically arranged so that the resulting image on the non-linear crystal 30 is of millimetre size, the method provides what is effectively a one-dimensional cross section of the ablated structure.

The pattern 36 recorded by the camera can easily be calibrated with depth information by moving the delay line a known amount and observing the corresponding change on the CCD camera.

As it can be deduced from FIG. 3b, a change in the delay of the probe beam 16 results in a displacement of the overlap region 34 across the image formed from the backscattered light 24. This corresponds to moving the virtual slit across the image of the ablated region 6 and can thus be used to map the cross section at various positions.

In a specific embodiment of the above technique, a non-linear crystal of BBO is applied. The two beams have their polarisation parallel to each other and perpendicular to the plane of incidence on the crystal (s-polarised). The crystal is oriented for the so-called and well-known phase matched type I second-harmonic generation for the two beams 16, 24 intersecting at an angle. With this specific choice of phase matching, the technique described will provide a cross-sectional profile across the ablated region 6 in the direction perpendicular to the plane spanned by the two beams 16, 24. In the absence of wave-plates in the optical set-up, this direction is parallel with respect to the polarisation of the light 14 incident on the surface 6 of the sample 7.

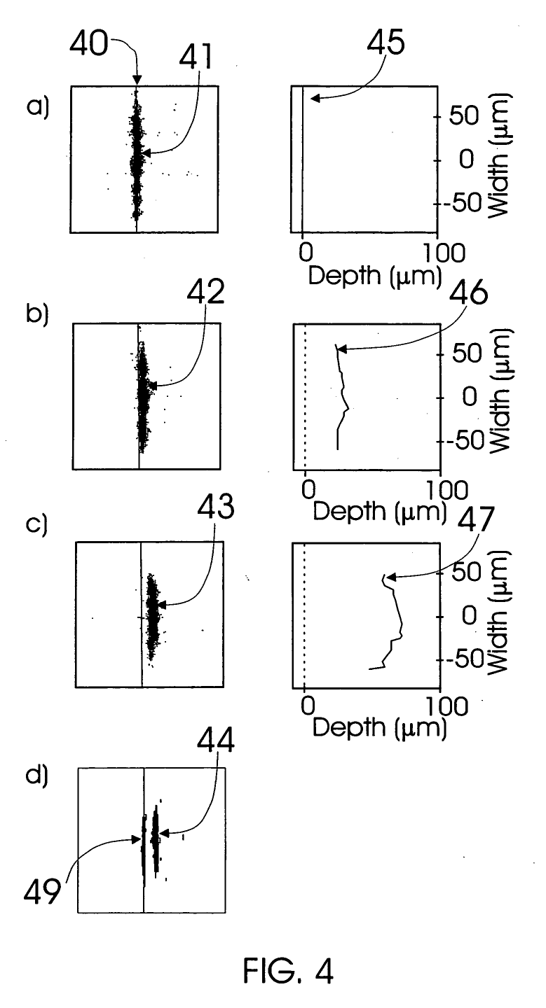

The left part of FIG. 4 shows a sequence of images obtained during machining of a stainless steel plate. The technique described above provides an image where the horizontal direction is associated with the temporal (or depth) co-ordinate and the vertical direction is a spatial co-ordinate along the polarisation direction, the position of which is selected by the specific delay of the timing pulse. In the images of FIG. 4, the delay is chosen so that a cross section through the middle of the ablated region is obtained. The horizontal axis illustrates flight time corresponding to depth, where shorter flight times are to the left on the image for the present choice of geometry. The vertical direction is associated with a spatial co-ordinate across the centre of an ablated hole.

On the image taken immediately after initiating the ablation, FIG. 4a, the sample is flat. Consequently all backscattered radiation traverses the same distance, resulting in a single vertical streak 41 on the image. On subsequent images, FIG. 4b and FIG. 4c, the central part of the laser beam has ablated material from the steel plate.

This forms a hole in the sample, and the light being scattered from this part of the sample will have a longer flight time to the gate and gives a signal 42, 43 which is to the right of the original streak 41. In the images, a thin vertical line 40 indicates the position of the unperturbed surface, and it can be seen that the backscattered light shown as a displaced streak 42 is now delayed with respect to this position, reminiscent of the longer distance to the bottom of the hole. In panel c), the displacement of the streak 43 relative to the non-displaced streak 41 becomes more pronounced.

The cross-sectional curves 45, 46, 47 shown in FIG. 4 right column are extracted from the left images. The scale of the curves 45, 46, 47 is obtained from a direct calibration. The depth calibration is obtained by moving the delay line 20 a certain amount and observing the horizontal shift of the streak 41, 42, 43. Similarly, the translation of an already formed hole a specific amount (roughly half a hole diameter) along the polarisation direction and observation of the vertical shift provides the spatial calibration (i.e. the magnification of the imaging system 26).

The initial alignment of the optical system used in the preferred embodiment of the present invention can be simplified by division into two steps. First, the two paths of the set-up for the two parts 14, 16 of the laser beam are aligned for identical optical path lengths. This can be done by replacing the lens 5 and sample 7 with a highly reflecting mirror. With such a set-up, it will be uncomplicated for those skilled in the art to align the set-up so that the production of second-harmonic light from the combined effect of the two beams is maximised. Now the two paths have the same length. Secondly, the lens 5 and a test sample 7 are reinserted and—if required—also the imaging lens system 26. If applicable, the imaging lens system 26 is then adjusted to produce an image of the sample surface at the required image plane, e.g., on the optical non-linear crystal 30. This can be done at low light levels and, as is clear to those skilled in the art, preliminary alignment is most easily performed at visible wavelengths after which only small corrections are needed upon changing to the laser wavelength.

In alignment and design of the imaging system, care must be taken to avoid the well-known classical imaging errors, also known as aberrations. In the present case, abberations have been minimised by using an aperture, in this case with a diameter of 8 mm, in front of the focusing lens 5 limiting the transmission of backscattered light to the central part of the lens 5. The effect of spherical abberations and coma is demonstrated in FIG. 4d, where a signal 44 has been recorded analogous to FIG. 4c, but without an aperture in front of the lens 5, which was a simple plano-convex lens. The aberration caused thereby resulted in false stray light 49 over the image formed by the backscattered light 24, in particular the backscattered light from the edges of the hole, giving rise to the artefact that a signal 49 remains at zero depth during all stages of the ablation.

Another solution to avoid the aberrations is to use aspherical optics for the lens 5 in accordance with the approach taken in standard light microscopes. This would preserve a large numerical aperture and thus a high lateral resolution in the imaging of the ablated region 6.

As was mentioned above, the depth resolution is related to the laser pulse duration. In this context, it is important to note that in the situation where the focusing lens 5 is employed to image an aperture onto the sample, a situation often used to obtain a roughly uniform intensity distribution on the sample, there will be a laser focus a few millimetres in front of the sample. If this focus is in atmospheric air, a significant pulse stretching is normally observed due to non-linear processes and in particular the so-called self-phase modulation. This has generally a negative effect on the laser ablation process, but in connection with the invention described here, it has the further consequence that the depth resolution is deteriorated. In order to avoid this effect, an inert gas with a low non-linear index of refraction (the so-called n₂) can be used. For instance, in obtaining the images of FIG. 4, helium gas was employed around the focal region of the lens.

In order to follow the laser ablation from pulse to pulse, it is necessary that an image be taken for each laser pulse. This means that the laser repetition rate and the video rate must be equal. While the ultrashort-pulse lasers used for laser machining typically have a repetition rate in the kilohertz regime, video rates are typically in the few tens of hertz region. In other words, in order to follow the machining from shot to shot, one must reduce the laser repetition rate and/or use high-speed cameras. Another possibility is to accept an image acquisition rate, which is lower than the laser repetition rate, but still high enough that the material removal can be resolved. In fact, a typical material-removal rate in the micromachining regime is on the order of 0.1 micrometers per laser pulse. Consequently, with the few micrometer depth resolution of the present invention, it will not be possible to observe the effect of less than a few tens of pulses. This implies that a video camera operating below 100 hertz can often be applied without loss of information. The images shown in the figures were all taken with a standard video camera and commercially available frame grabber.

Although the average depth does not change significantly over the number of laser shots relevant for kilohertz-repetition-rate lasers and standard video rates, obtaining the image as an average over several tens of laser shot has another consequence: An investigation at a reduced laser repetition rate shows that the geometrical information revealed by a single laser pulse is influenced by the specific situation on the surface 6 left by the previous laser pulse. Specifically, it can be seen that small particles left on the surface (debris) change the image from laser shot to laser shot. When averaging over a few tens of laser pulses (i.e. when operating the laser in the standard kilohertz-repetition-rate regime), the effect of these shot-to-shot fluctuations is an additional smearing of the depth profile. The ˜20 μm smearing (full-width at half max) in the depth co-ordinate on the images in FIG. 4 is composed of two roughly equal contributions: The laser, pulse pulse-duration of 100 fs corresponds to cT/2=15 μm and in addition, the shot-to-shot fluctuations contribute a similar amount.

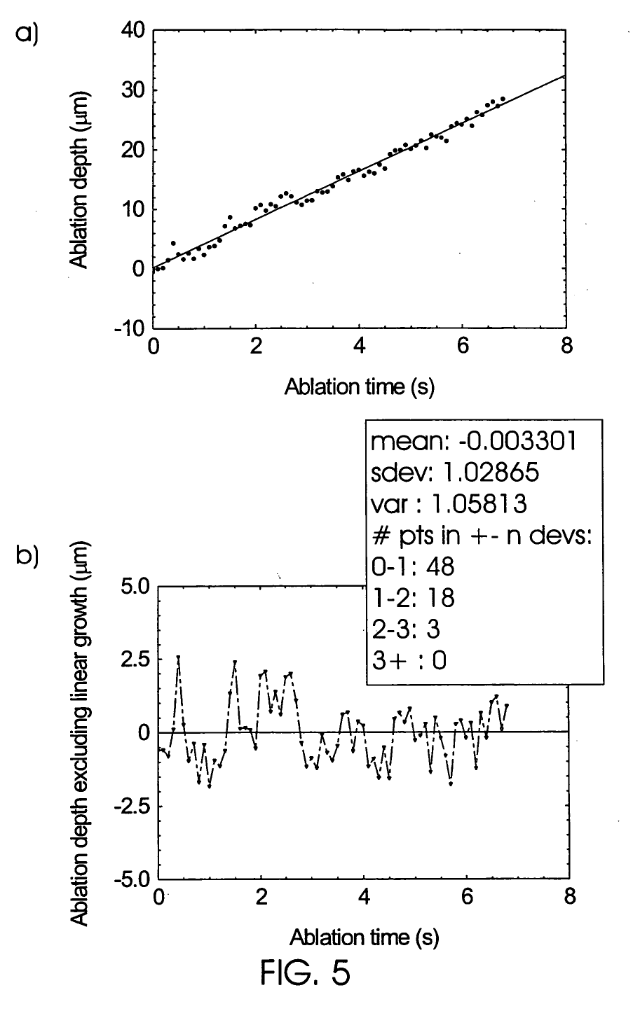

It is important to note that the above depth smearing is the full-width at half-max value. The depth resolution is determined by the accuracy with which the position of this distribution can be determined. This accuracy depends on the statistical significance of the measurement, but is typically a small fraction of the full-width at half-max width. FIG. 5a shows the measured depth versus ablation time for a stainless steel plate determined from a sequence of images as presented in FIG. 4. As can be seen, the ablation rate is to a good approximation constant and the depth versus time is well fitted by a straight line. In FIG. 5b, this linear term has been eliminated from the measured depth to allow a study of the accuracy of the measurement. The points scatter with a standard deviation as small as 1 μm, which is only 5% of the full width at half max mentioned above.

The system described in the present invention can hence produce on-line information about the profile of an area subjected to laser ablation. It is clear that this information can be used to control the machining process. The most obvious use is to apply the invention to stop the machining of a sample at a given pre-determined depth. This is obviously useful for high-accuracy three-dimensional micromachining and for some applications in laser surgery.

A second application is to use the depth profile as a feedback system for adjusting the position of the focusing device (e.g. lens) used to focus or image a mask onto the sample. When machining to a significant depth is required, in order to preserve lateral (transverse) dimensions, it may be necessary that the lens-sample distance be adjusted so that the part subjected to machining (e.g. the bottom of a hole) is always kept at the right distance. This can be achieved by using the present invention.

In some applications of laser machining, translation of the sample relative to the laser is employed. In this manner, material can be removed from an extended area. This method is sometimes referred to as laser milling. The present invention facilitates laser milling to a well-controlled depth. The depth profile is fed back to the scanning system to adjust the velocity of the sample (or laser) so that a specific final depth is obtained. Since this method mostly relies on the geometry along the axis of translation, the optical gate needs only to allow one-dimensional imaging.

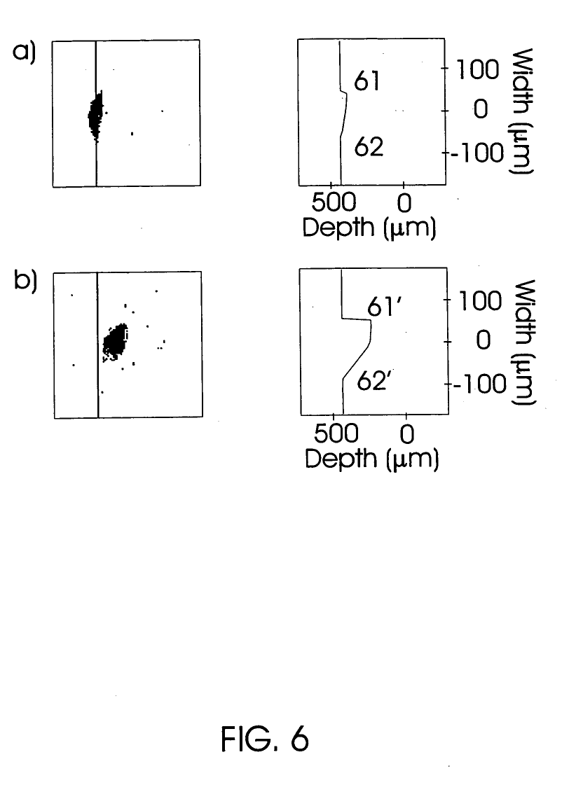

FIG. 6 shows two images obtained during laser milling. In both images in FIG. 6, the sample is moved upward relative to the image. The slope in the depth profiles illustrates the different amounts of material removed from those regions leaving the laser focus 61 and just entering the laser focus 62. FIG. 6a shows an image obtained during a fast translation of the sample (milling to a shallow depth 61), while FIG. 6b corresponds to a slower sample translation (milling to larger depth 61′). The feedback system is based on images/profile measurements like this. In the images of FIG. 6, the pulse energy was kept constant and only the sample-translation speed was changed. Based on the feedback from images, it will also be possible to gradually reduce the pulse energies to reduce the ablation rate. This may be useful for accurate fabrication of fine details.

The above description devised an apparatus for producing cross-sectional information along a specific axis across the laser-ablated region. Since this direction is dictated by the axis being perpendicular to the plane spanned by the two beams entering the non-linear crystal, this axis cannot easily be rotated. One can, however, easily add another axis simply by duplicating the system so that another optical gate is applied. The optical arrangement would then be arranged so that the signal and timing beams for this gate span a different plane and thus select a different direction across the laser-ablated region.

A special application of the invention is in the laser machining of uneven samples. As mentioned above, the quality of the produced structures depends critically on an accurate control of the lens-sample distance. If an uneven sample is translated during machining, this distance must be controlled. If the depth profiling method is applied, it is possible to determine the varying distance during machining and the signals will be sufficient for adjusting both the scan velocity (feed back from the slope of the ablated surface) and the focusing geometry (feed back from the level of the surface at the incoming edge). This application can be very useful for medical applications of laser ablation, where the sample subjected to the laser will in general be of a complicated geometrical shape.

In the descriptions above, the emphasis has been on the extraction of a cross-sectional profile. For the drilling of through-holes, this information can still be valuable, since it can be used to ensure a desired profile of the bottom of the hole prior to penetration of the sample. This may be important to obtain the desired geometry of a through-hole.

It should also be noted that recording of the cross-sectional information during the drilling of a through hole does in fact contain information about the geometry of the hole formed. Specifically, the width of the streak versus depth provides the width of the hole versus depth, i.e. the so-called taper of the hole. This property can be very important for the hole characteristics, e.g. for their use as nozzles in various applications.

Note that contrary to the cross-sectional information, which is obtained on the fly, the above-mentioned information about the taper of a through hole is only obtained by accumulation of the entire sequence of images. More precisely, recording of all images down to a certain depth provides the taper of the hole down to that depth. In other words, the entire taper is revealed after the hole is completed. However, the information obtained during the drilling can be used to adjust the machining parameters to optimise the desired taper. In fact, from the entire sequence of images not only the taper of the hole can be extracted, but the shape of the side wall of the hole can be reconstructed.

In some methods designed to optimise through-hole drilling, a laser spot size smaller than the desired hole diameter is applied; the laser spot is then moved around on the sample to obtain the best possible geometry. Independent of the choice of method (as the so-called trepanning- or helical-drilling techniques), the method for obtaining geometrical information can, however, still be used. Of course, moving the laser spot during machining means that the geometrical information retrieved on the fly is related to different points on the surface, but recording of the geometrical information together with the well-known co-ordinates of the areas subjected to ablation will still provide the entire profile of the laser-ablated region. Specifically, in the limit that the laser spot size is small compared to the size of the structures formed, the imaging embodiment of the present invention may not be needed: the depth at the point struck by the laser is recorded, and as the laser spot is moved around on the sample, the depth profile is acquired point by point using a scanning probe technique.

Interestingly, this particular application—to measure the taper or the side wall profile of a through hole—is less critically dependent on the pulse duration of the laser. Even for a laser with picosecond pulse duration, the taper information is valuable: it will provide the width of the hole with a ˜100 micrometer depth-resolution, and since the taper normally develops over these depth-scales, not much information is lost.

Provided the reflectivity of a sample is sufficient below the damage threshold, it is clear that the distance measurement described in the present invention can be applied as a surface profiling system prior to any machining. For instance, one could envision a situation, where selective machining can be applied: a profile is acquired and certain regions, e.g. protrusions, are then machined by the laser until a desired profile is obtained. Another application of this pre-machining profiling is to be able to target a laser-ablated region in a subsequent pass. It is often found that the best machining results are obtained by repeated machining, where each pass removes only a thin layer. With the present invention one will be able to lock the machining laser to an already existing structure on the surface as, e.g., a slot from previous laser milling.

A special employment of the invention is in laser ranging to objects that have a very small reflectivity. Such objects are difficult to observe by conventional laser ranging methods. With the present invention one can apply a few shots above the threshold for plasma formation, which will allow a determination of the distance to high accuracy, as described above. In many applications the structural changes induced by a few laser shots (i.e. material removal on the order of 1 micrometer) will be unimportant.

Another special application of the invention is in the machining of transparent materials. As has been demonstrated in several experimental studies, it is possible to make an arrangement with ultrashort laser pulses, which leads to machining (or more generally changes of material properties) only in the inside of a transparent medium. In such an arrangement, it is useful to be able to control the distance to the surface of the medium very accurately. This can be done by collecting the light, which is reflected from the surfaces and performing a high-resolution measurement of its temporal structure. This information can be converted to distance using the method described above and thus can be used to control the machining depth. In one embodiment, both the light from the surface and the backscattered light from the machined region inside the medium are detected, thus providing a direct measurement of the depth beneath the surface of the machined region. Apart from applications for modifying optical materials properties (e.g. for the writing of wave-guides), this method could be useful for eye surgery.

While the above demonstration was made in the specific embodiment of a femtosecond laser based on titanium-sapphire technology, it is important to note that the method described in this patent does not at all depend on this choice of implementation. The same remark is valid with respect to the specific choice of the optical gating method. To clarify this statement, the necessary source and gate characteristics are summarised below. In the future it is very likely that the rapid technical developments in optical and electronic engineering will produce new laser systems and ultrafast gates, which will fulfil these requirements.

The previous studies of laserablation with ultrashort pulses have covered a large range of wavelengths, pulse durations, and pulse energies. The method described here is in general applicable with all of these conditions. The two constraints on the laser source are (i) that the depth resolution of the present technique, as described in detail above, is dependent on the pulse duration of the light and (ii) that the laser delivers enough energy that some fraction of the light can be used for the optical gating. In practice this means that the method is of highest interest for laser pulses with a duration in the 100-femtosecond range or shorter and pulse energies above ˜10 microjoules. This is not a very strict demand to future laser sources. One very interesting area of development in this connection is in fibre-based amplified ultrashort-pulse laser systems. As described in U.S. Pat. No. 6,014,249, this technique could potentially lead to very stable and high-efficiency lasers.

The images of the enclosed FIGS. 4 and 6 were all taken using an optical gate based on second-harmonic generation. This was merely done for the purpose of illustration. As it is clear to those skilled in the art, many other frequency-mixing techniques can be applied. In general, the only restriction is that the appropriate materials (non-linear medium) for the desired process exist, and this is an area of ongoing research. In addition, other optical gating mechanisms are available (e.g. a Kerr gate), and technical developments may lead to improved performances of these alternative optical gates. Finally, the technical advancements in ultrafast electronics (confer, e.g., a streak camera) may at some point lead to the development of a non-optical gate, which is fast enough that the present technique can be implemented with an electronic gate instead of the optical gate.

Based on the rapid advances in fibre technology, it is also worth noting that all of the basic elements needed for the present method are in principle available as fibre-optic elements (beam-splitter, polariser, wave-plate, delay-line, and non-linear medium). It is therefore quite possible that the technique be implemented (at least in the non-imaging configuration) as an all-fibre set-up. This implementation is especially interesting in connection with a fibre-based laser source.

1. Method for measuring material removal during laser irradiation wherein an ultrashort laser pulse is focused in a region of a sample for removal of material from said region and wherein scattered radiation is collected from said region, wherein the method further comprises determination of geometric information of said sample region from said scattered radiation,
wherein
said collected radiation is scattered radiation from said ultrashort laser pulse, and
flight-time information of said laser pulse is obtained and this flight-time information is converted to distance to obtain depth information of said region.

2. Method according to claim 1, wherein said laser radiation is splitted into at least a first part and a second part, where the first part constitutes said ultrashort laser pulse undertaking said removal of material, and the second part provides a timing signal for the flight-time determination.

3. Method according to claim 2, wherein said timing signal controls an optical gate for selecting the backscattered light with a specific flight time.

4. Method according to claim 3, wherein said optical gate is based on non-linear frequency mixing.

5. Method according to claim 4, wherein said non-linear frequency mixing is performed in a non-collinear geometry.

6. Method according to claim 1, wherein said scattered radiation is recorded to obtain cross-sectional information of said region.

7. Method according to claim 6, wherein said cross-sectional information is obtained by time resolved imaging of said backscattered radiation onto a detector, wherein the cross section is in a plane normal to the direction of travel of said ultrashort laser pulse.

8. Method according to claim 7, wherein said obtaining of cross-sectional information comprises imaging of said backscattered light on a non-linear medium in spatial and temporal overlapping conditions with said second part, whereby a pattern is produced in said medium by non-linear frequency mixing, wherein the pattern is indicative of said cross-sectional information, and wherein the cross section is in a plane parallel to the direction of travel of said ultrashort laser pulse.

9. Method according to claim 1, wherein an inert gas with a low non-linear index of refraction is employed around the focus of said laser pulse in order to optimise the resolution of said geometric information, preferably depth resolution.

10. Method according to claim 1, wherein said scattered radiation originates from at least one from the group consisting of
a reflecting surface,
a mostly absorbing surface, where the scattering is enhanced by a transient high reflectivity induced by the laser,
diffuse scattering on a surface of a sample,
scattering on a plasma during or after formation, where the plasma originates on a surface,
scattering on a plasma during or after formation, where the plasma originates inside a transparent sample.

11. Method according to claim 6, wherein said cross-sectional information is used for at least one from the group consisting of
adjusting the scan rate during laser machining,
adjusting the position of the irradiated sample to maintain optimum focusing conditions on the sample region subjected to machining during translation.

12. Method according to claim 1, wherein said scattered radiation originates from scattering on a plasma inside a transparent sample, and wherein also radiation reflected from the outer surface of the transparent sample is collected for determining the distance to the outer surface of the transparent sample in order to measure the exact position of the plasma inside the sample.

13. A method according to claim 1, wherein the method comprises adjusting the position of the irradiated sample to maintain optimum focusing conditions on the area subjected to machining during the removal of material.

14. A method according to claim 11, wherein the method comprises laser surgery or eye surgery.

15. A method according to claim 12, wherein the method comprises eye surgery.

16. A method according to claims 1, wherein the method comprises reconstruction of a side wall profile of a laser machined hole after repeated laser irradiation.
